(19) United States
(12) Patent Application Publication
(10) Pub. No.: US 2026/0014019 A1
(43) Pub. Date: Jan. 15, 2026

(54) HEATING ELEMENT FOR HAND WARMER AND HAND WARMER

(71) Applicant: Na Zhou, Dongguan City (CN)

(72) Inventor: Yong Zhou, Dongguan City (CN)

(21) Appl. No.: 19/257,609

(22) Filed: Jul. 2, 2025

(30) Foreign Application Priority Data
Jul. 10, 2024 (CN) 202421630615.8

(51) Int. Cl. A61F 7/00; H05B 3/36

(52) U.S. Cl. CPC A61F 7/007; H05B 3/36; A61F 2007/0036; A61F 2007/0071; A61F 2007/0078; H05B 2203/003

(57) ABSTRACT

A heating element for a hand warmer, including: a base body, at least three wiring terminals, a first coil, and a second coil. The first coil and the second coil are fixedly spaced apart on the base body, and the at least three wiring terminals are electrically connected to two ends of each of the first coil and the second coil.

### CROSS-REFERENCE TO RELATED APPLICATIONS

The present application claims the priority of Chinese patent application No. 202421630615.8, filed on Jul. 10, 2024, and the entire contents of which are hereby incorporated by reference in its entirety.

### TECHNICAL FIELD

The present disclosure relates to the technical field of heating equipment, and more specifically to a heating element for a hand warmer.

### BACKGROUND

The heating element for a hand warmer is configured to convert electrical energy into thermal energy, enabling the hand warmer to provide warmth to hands in cold conditions. Typically powered by a built-in rechargeable battery, the hand warmer generates heat through electrical current flowing through the heating element. However, in the related art, the heating element generally incorporates only a single coil, resulting in a fixed temperature setting. Given the divergent thermal sensitivity and preferences among users, this single temperature setting fails to accommodate diverse needs, thereby compromising both functionality and user experience.

The above content is provided solely for the purpose of assisting in understanding the technical solutions of the present disclosure and does not constitute acknowledgment that the above content constitutes prior art.

### SUMMARY OF THE DISCLOSURE

The present disclosure provides a heating element for a hand warmer, including: a base body, at least three wiring terminals, a first coil, and a second coil;
wherein the first coil and the second coil are fixedly spaced apart on the base body, and the at least three wiring terminals are electrically connected to two ends of each of the first coil and the second coil.

In some embodiments, the base body is configured in a plate-like shape, with the first coil and the second coil located on opposite surfaces of the base body.

In some embodiments, the base body includes a first surface and a second surface opposite the first surface; the first surface includes a connection region and a heating region connected to the connection region; the first coil is distributed in the heating region, and the at least three wiring terminals are fixed in the connection region;
the at least three wiring terminals include a first terminal and a common terminal, wherein the first terminal and the common terminal pass through the first surface and the second surface to connect the two ends of the second coil; the second coil is arranged in a corresponding heating region on the second surface facing the heating region and in a corresponding connection region on the second surface facing the connection region, while avoiding positions where the first terminal and the common terminal are located.

In some embodiments, the second coil on the corresponding connection region extends serpentine along a first direction, and the second coil on the corresponding heating region extends serpentine along a second direction, with the first direction perpendicular to the second direction.

In some embodiments, the second coil includes a first segment and a second segment to the first segment; the first segment is connected to the first terminal, and the second segment is connected to the common terminal; the first segment and the second segment are symmetrically arranged along the second direction.

In some embodiments, the first coil includes a third segment and a fourth segment extending serpentine along the second direction, with the third segment and the fourth segment distributed and connected along the first direction;
the heating element further includes a temperature sensor, disposed on the first surface; wherein one of the two ends of each of the first coil and the second coil and one of two ends of the temperature sensor are connected to the common terminal.

In some embodiments, the at least three wiring terminals further include a second terminal and a third terminal; the third segment is connected to the second terminal, and the fourth segment is connected to the common terminal; the two ends of the temperature sensor are connected to the third terminal and the common terminal.

In some embodiments, a surface of at least one of the first coil and the second coil is covered with an adhesive layer.

The present disclosure further provides a hand warmer, including a housing, a control board, and the heating element as above; wherein the heating element and the control board are both arranged within the housing, and the heating element is electrically connected to the control board.

In some embodiments, the housing includes a heat-conducting shell and an inner shell connected to the heat-conducting shell; the heat-conducting shell covers an outer wall of the inner shell, and the heat-conducting shell and the inner shell enclose to define a heating cavity; the heating element is disposed within the heating cavity; the inner shell defines a mounting cavity, and the heating cavity is in communication with the mounting cavity; the control board is disposed within the mounting cavity, with the at least three wiring terminals facing the inner shell.

### BRIEF DESCRIPTION OF THE DRAWINGS

To more clearly illustrate the technical solutions of the embodiments of the present disclosure or the related art, the following is a brief introduction to the drawings used in the description of the embodiments or the related art. It is obvious that the drawings described below are only some embodiments of the present disclosure. For those skilled in the art, other drawings can be obtained based on the structures shown in these drawings without creative labor.

FIG. 1 is a structural schematic view of a first surface of a heating element for a hand warmer according to some embodiments of the present disclosure.

FIG. 2 is a structural schematic view of a second surface of the heating element, as shown in FIG. 1, for a hand warmer.

FIG. 3 is a structural schematic view of a hand warmer according to some embodiments of the present disclosure.

FIG. 4 is an exploded schematic view of a hand warmer according to some embodiments of the present disclosure.

FIG. 5 is a cross-sectional schematic view of the hand warmer as shown in FIG. 1.

### DETAILED DESCRIPTION

The technical solutions in the embodiments of the present disclosure will be described clearly and completely in the following in conjunction with the accompanying drawings in the embodiments of the present disclosure, and it is obvious that the described embodiments are only a part of the embodiments of the present disclosure and not all of the embodiments. Based on the embodiments in the present disclosure, all other embodiments obtained by those skilled in the art without creative labor fall within the scope of the present disclosure. In addition, the technical solutions of different embodiments can be combined with each other, but they must be based on what is achievable by those skilled in the art. When the combination of technical solutions is contradictory or unachievable, such combination of technical solutions shall be deemed not to exist and shall not be included in the scope of protection claimed by the present disclosure.

It should be noted that when the embodiments of the present disclosure involve directional indications (such as up, down, left, right, forward, back . . . ), the directional indications are only intended to explain a relative positional relationship, a movement, etc. between the various components in a particular attitude. When the particular attitude changes, the directional indications are also changed accordingly.

In addition, when the embodiments of the present disclosure contain descriptions involving “first”, “second”, etc., the descriptions of “first”, “second”, etc. are intended only for descriptive purposes, and are not to be construed as indicating or implying their relative importance or implicitly specifying the number of the indicated technical features. That is, a feature defined as “first” or “second” may include at least one such feature either explicitly or implicitly. In addition, the meaning of “and/or” in the whole text is to include three concurrent solutions. For example, “A and/or B” includes an A solution, a B solution, and a solution in which A and B are satisfied at the same time.

The present disclosure proposes a heating element 100 for a hand warmer.

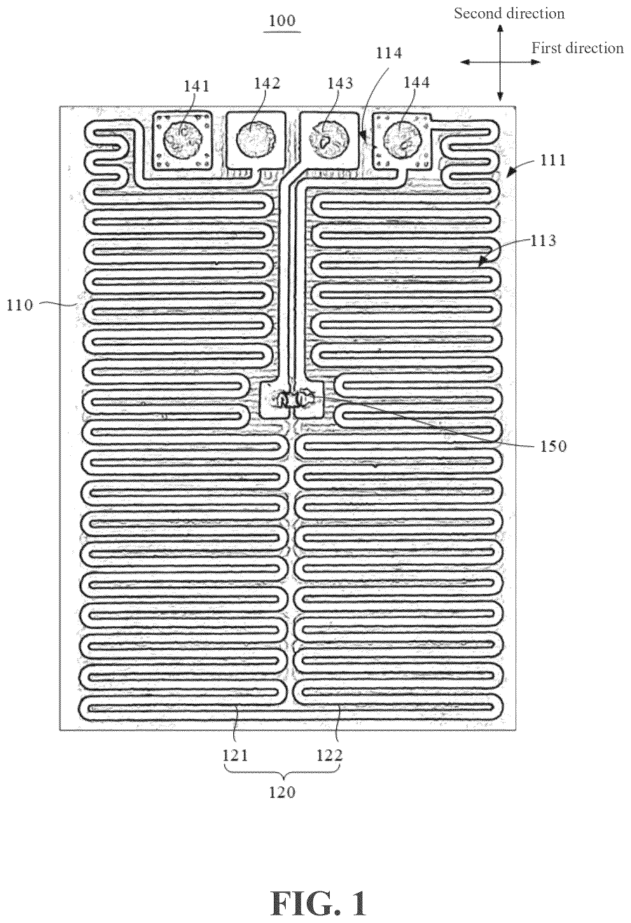

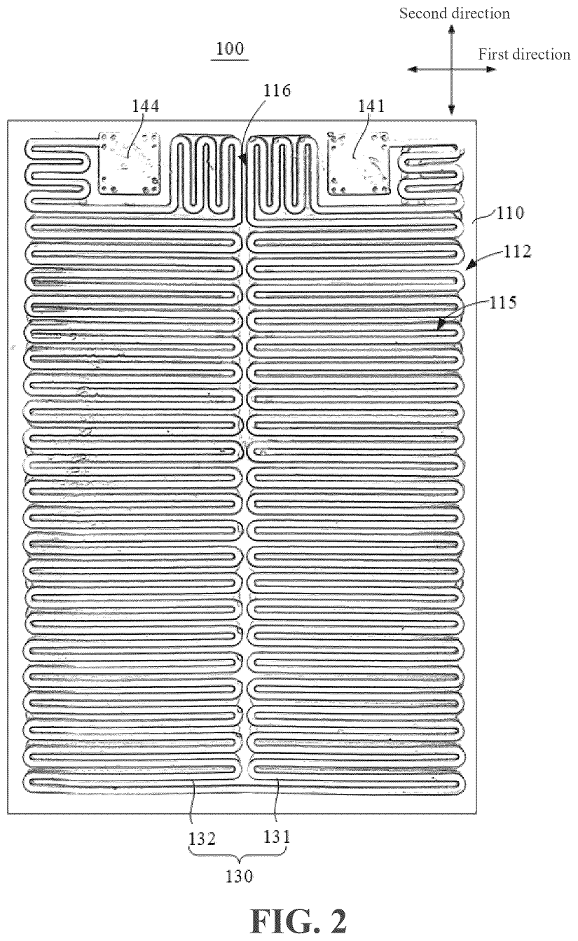

In the embodiments of the present disclosure, referring to FIGS. 1 and 2, the heating element 100 includes a base body 110, at least three wiring terminals, a first coil 120, and a second coil 130. The first coil 120 and the second coil 130 are fixedly spaced apart on the base body 110, and the at least three wiring terminals are electrically connected to two ends of each of the first coil 120 and the second coil 130.

The heating element 100 of the present disclosure, by providing the first coil 120 and the second coil 130, may achieve different power outputs by selectively allowing current to flow through the first coil 120, the second coil 130, or both the first coil 120 and the second coil 130 simultaneously, thereby enabling multi-level temperature regulation. Users may select an appropriate temperature setting based on their individual temperature sensitivity and preferences, thereby enhancing the user experience. Further, the first coil 120 and the second coil 130 are fixedly spaced apart on the base body 110 to ensure the uniformity of heating of the heating element 100, avoiding the local overheating problem that may occur in traditional single coils, thereby improving the safety and comfort of the hand warmer 200.

In some implementations, the heating element 100 may include three wiring terminals, which are configured to connect wires, and the wires are connected to a control board of the hand warmer to ensure electrical conductivity. Each of the first coil 120 and the second coil 130 are connected to corresponding two wiring terminals, and one end among the two ends of each of the first coil 120 and the second coil 130 is connected to a common terminal 144, thereby connecting the first coil 120 and the second coil 130 in parallel. Through a control circuit, current can flow through the first coil 120, the second coil 130, or both simultaneously, thereby enabling multi-level temperature regulation.

It should be noted that the resistance values of the first coil 120 and the second coil 130 may be the same or different. When the resistance values of the first coil 120 and the second coil 130 are the same, two temperature settings may be achieved, and the first coil 120 and the second coil 130 may be used alternately, thereby reducing the rate of coil aging and extending the service life of the heating element 100. When the resistance values of the first coil 120 and the second coil 130 are different, the heating element may have three resistance values, enabling the hand warmer to output three power levels and achieve three-stage temperature regulation, thereby enhancing the functionality of the heating element 100. The specific resistance value ranges may be set according to actual requirements and are not limited herein. Additionally, the first coil 120 and the second coil 130 may be located on the same surface of the base body 110 or on opposite surfaces of the base body 110, with no restrictions imposed here.

In some embodiments, the base body 110 is configured in a plate-like shape, with the first coil 120 and the second coil 130 located on opposite surfaces of the base body 110.

The base body 110 is configured in the plate-like shape, enabling the heating element 100 to closely adhere to an inner surface of a housing 210 of the hand warmer 200, ensuring more efficient heat conduction between the heating element 100 and the housing 210, thereby improving the overall heating efficiency of the hand warmer 200. Additionally, the plate-shaped base body 110 helps maximize the limited internal space of the hand warmer 200 and increase the heat dissipation area of the heating element 100. This allows the heating element 100 to maintain an effective heating area while minimizing interference with other components inside the housing 210 and reducing space occupation.

The first coil 120 and the second coil 130 are respectively fixed to the opposite sides of the base body 110, enabling uniform heat distribution and preventing local overheating or overcooling, thereby enhancing the comfort and safety of the hand warmer 200. Additionally, since the first coil 120 and the second coil 130 are distributed on opposite sides of the base body 110, the current may be controlled to flow through each coil individually or through both coils simultaneously, enabling multi-temperature regulation of the hand warmer 200 to meet the temperature preferences of different users. When current flows through both coils simultaneously, the heating element 100 can perform dual-sided heating, significantly improving the heating efficiency of the hand warmer 200 and enabling it to reach the desired temperature in a shorter time. Additionally, by arranging the first coil 120 and the second coil 130 on opposite sides of the base body 110, direct contact between the coils may be avoided, thereby reducing the risk of short circuits or other electrical faults and thus enhancing the safety of the hand warmer 200.

Specifically, the base body 110 includes a first surface 111 and a second surface 112 opposite the first surface 111. The first surface 111 includes a connection region 114 and a heating region 113 connected to the connection region 114. The first coil 120 is distributed in the heating region 113, and the at least three wiring terminals are fixed in the connection region 114. The clear division between the heating region 113 and the connection region 114 on the first surface 111 ensures that the coils and wiring terminals are arranged in an orderly manner, avoiding space wastage.

The at least three wiring terminals include a first terminal 141 and a common terminal 144, where the first terminal 141 and the common terminal 144 pass through the first surface 111 and the second surface 112 to respectively connect the two ends of the second coil 130. The second coil 130 is arranged on the second surface 112 facing the heating region 113 (i.e., a corresponding heating region 115) and the connection region 114 (i.e., a corresponding connection region 116), while avoiding positions where the first terminal 141 and the common terminal 144 are located. The second coil 130 on the second surface 112 is arranged corresponding to the heating region 113 and the connection region 114 and avoids the wiring terminals, thereby optimizing the internal structural layout while increasing the coverage area of the second coil 130, and thus increasing the heat dissipation area of the second surface 112.

It should be noted that the shape of the base body 110 may be ring-shaped, circular, oval, square, or other shapes, which is not limited herein.

By passing the first terminal 141 and the common terminal 144 through both sides of the base body 110, the first coil 120 and the second coil 130 are independently connected, simplifying the circuit design and reducing circuit complexity. This further ensures that the electrical connection of the second coil 130 avoids the positions of the first coil 120 and other terminals, reducing electrical interference between coils and improving circuit stability. Additionally, the above passing-through design makes the terminal positions more reasonable, avoiding overlap between terminals and coils, thereby optimizing internal space layout and facilitating heat dissipation and heat conduction.

Furthermore, the second coil 130 on the corresponding connection region 116 extends serpentine along a first direction, while the second coil 130 on the corresponding heating region 115 extends serpentine along a second direction, with the first direction perpendicular to the second direction.

In the embodiments, the base body 110 has a flexible strip-like structure, with the first direction being a width direction of the base body 110 and the second direction being a length direction of the base body 110. The second coil 130 extends in a serpentine pattern along the first direction in the corresponding connection region 116 and along the second direction in the corresponding heating region 115. This arrangement ensures uniform distribution of the coils on the base body 110, thereby avoiding local overheating or cooling issues, and enabling more uniform overall heating of the heating element 100. Additionally, the serpentine extension design may maximize the coil layout area within the limited space of the base body 110, fully utilizing the surface area of the base body 110, thereby enhancing the heating capacity of the heating element 100. Furthermore, the second coil 130 extends along the first direction in the corresponding connection region 116, enabling the coil to maximize coverage of the corresponding connection region 116 and increase the heating area of the second coil 130. Additionally, the symmetrical serpentine extension design helps distribute thermal stress during heating and cooling processes, reducing the risk of coil breakage or deformation, thereby extending the service life of the heating element 100.

Furthermore, the second coil 130 includes a first segment 131 and a second segment 132 connected to the first segment 131. The first segment 131 is connected to the first terminal 141, and the second segment 132 is connected to the common terminal 144. The first segment 131 and the second segment 132 are symmetrically arranged along the second direction.

In this configuration, the first segment 131 and the second segment 132 of the second coil 130 are respectively connected to the first terminal 141 and the common terminal 144, and the two segments of the coil are symmetrically arranged. This symmetrical design ensures the stability of electrical connections, reduces the risk of uneven current distribution, and improves overall electrical performance. Furthermore, by symmetrically arranging the second coil 130, the space of the base body 110 may be utilized more effectively, increasing the arrangement density and heating area of the second coil 130, thereby enhancing the overall heating capability of the hand warmer 200.

Furthermore, the first coil 120 includes a third segment 121 and a fourth segment 122 extending in a serpentine pattern along the second direction, with the third segment 121 and the fourth segment 122 distributed and connected along the first direction. This arrangement ensures uniform distribution of the coils on the base body 110, avoiding issues such as local overheating or cooling, thereby achieving more uniform heating of the heating element 100 as a whole.

Furthermore, the heating element 100 further includes a temperature sensor 150, which is disposed on the first surface 111. An end of the first coil 120, the second coil 130, and the temperature sensor 150 are all connected to the common terminal 144.

In the embodiments, the temperature sensor 150 is adopted with a Negative Temperature Coefficient (NTC) temperature sensor, which can accurately monitor the temperature of the heating element 100 and adjust the power of the heating element 100 through the control circuit. In other words, when the temperature does not reach a target temperature, the control circuit promptly switches to high power to ensure the stability of the temperature of the heating element 100; when the target temperature is reached, the control circuit promptly switches to low power to enhance the battery life of the hand warmer 200. The temperature sensor 150 is disposed on the first surface 111 between the third segment 121 and the fourth segment 122, enabling more precise detection of the coil's temperature and, consequently, the heating element's temperature, thereby ensuring that the hand warmer 200 operates within an appropriate temperature range to prevent overheating or insufficient heating. Through the feedback from the temperature sensor 150, the control circuit can precisely regulate the current flowing through the first coil 120 and the second coil 130, achieving precise temperature control to meet users' different temperature requirements. In other embodiments, the temperature sensor 150 may be disposed at other positions on the first surface 111, without limitation.

Specifically, the at least three wiring terminals further include a second terminal 142 and a third terminal 143. The third segment 121 is connected to the second terminal 142, and the fourth segment 122 is connected to the common terminal 144. The two ends of the temperature sensor 150 are respectively connected to the third terminal 143 and the common terminal 144.

In this configuration, the two ends of the first coil 120 are connected to the second terminal 142 and the common terminal 144, respectively, while the two ends of the second coil 130 are connected to the first terminal 141 and the common terminal 144, respectively, and the two ends of the temperature sensor 150 are connected to the third terminal 143 and the common terminal 144, respectively, making the circuit layout more reasonable and optimized, reducing wire crossing and interference, and achieving parallel connection of the first coil 120, the second coil 130, and the temperature sensor 150, enabling the control circuit to independently control any one of them, thereby making the control circuit's control over the heating element 100 more flexible. Specifically, the first terminal 141, the second terminal 142, and the third terminal 143 can each serve as a positive terminal, while the common terminal 144 serves as a negative terminal. The first terminal 141, the second terminal 142, the third terminal 143, and the common terminal 144 are all connected to wires, which are configured to be connected to the control board of the hand warmer 200 to ensure circuit conductivity.

Specifically, the operating principle of the hand warmer 200 is as follows.

When the user turns on the hand warmer and selects a mode, high power is first output to quickly raise the temperature of the heating element 100 to the target temperature. The temperature sensor 150 continuously monitors the temperature of the heating element 100. When the target temperature is reached, a signal is transmitted to the control circuit, which receives the signal and outputs low power to enhance the battery life of the hand warmer 200. When the temperature sensor 150 detects that the temperature of the heating element 100 is below the target temperature, it transmits a signal to the control circuit. The control circuit receives the signal and outputs intermediate power to ensure that the heating element 100 reaches the target temperature while maintaining battery life. When the heating element 100 still does not reach the target temperature after a certain period of intermediate power output, the hand warmer 200 outputs high power to ensure that the heating element 100 reaches the target temperature.

It should be noted that different modes correspond to different target temperatures. In other words, a first mode of the hand warmer 200 corresponds to a first target temperature, a second mode corresponds to a second target temperature, and a third mode corresponds to a third target temperature. When the hand warmer 200 outputs high power, the current flows through both the first coil 120 and the second coil 130. When the hand warmer 200 outputs intermediate power, the current flows through a coil with lower resistance. When the hand warmer 200 outputs low power, the current flows through a coil with higher resistance.

Furthermore, a surface of the first coil 120 and/or the second coil 130 is covered with an adhesive layer (not shown), which is configured to bond the heating element 100 to the housing 210 of the hand warmer 200. The material of the adhesive layer may be a thermally conductive adhesive, ensuring that the heating element 100 is in close contact with the housing 210 while improving heat transfer efficiency. During production, the first coil 120 and second coil 130 coated with the adhesive layer are installed inside the housing 210. The adhesive layer forms a strong bond during heating or curing, thereby ensuring that the coils are securely fixed to the housing 210.

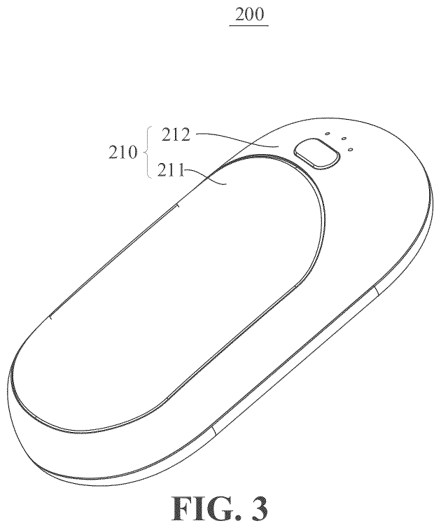

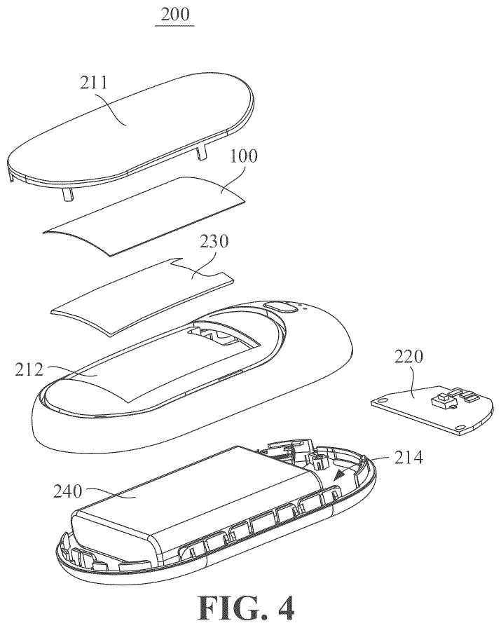

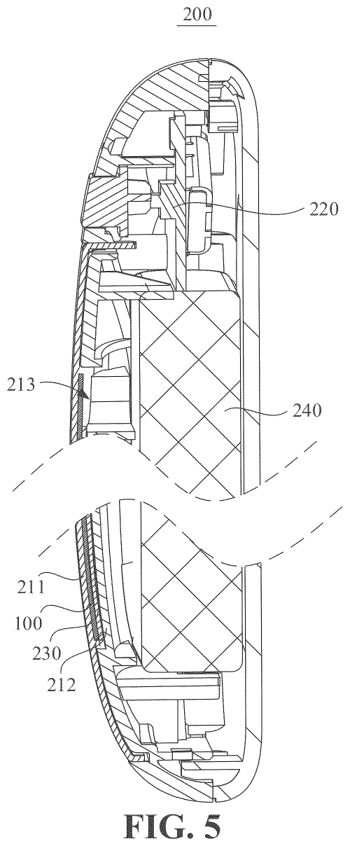

Referring to FIGS. 3 to 5, the present disclosure further proposes a hand warmer 200, which includes a housing 210, a control board 220, and a heating element 100. The specific structure of the heating element 100 may be referred to the above embodiments. Since the hand warmer 200 is adopted with all the technical solutions of the above embodiments, it possesses all the beneficial effects of the technical solutions of the above embodiments, which are not repeated herein. Specifically, the heating element 100 and the control board 220 are both arranged within the housing 210, which provides protective coverage for the heating element 100 and the control board 220. The heating element 100 is electrically connected to the control board 220 via wires connected to the wiring terminals, enabling the control board 220 to control the electrical circuit of the heating element 100.

Specifically, the housing 210 includes a heat-conducting shell 211 and an inner shell 212 connected to the heat-conducting shell 211. The heat-conducting shell 211 covers an outer wall of the inner shell 212, and the heat-conducting shell 211 and the inner shell 212 enclose to define a heating cavity 213. The heating element 100 is disposed within the heating cavity 213. The inner shell 212 defines a mounting cavity 214, and the heating cavity 213 is in communication with the mounting cavity 214. The control board 220 is disposed within the mounting cavity 214, with the at least three wiring terminals facing the inner shell 212, thereby allowing the wires of the control board 220 to pass directly through a communication hole between the heating cavity 213 and the mounting cavity 214. In this way, wire crossing and complex wiring may be reduced, thereby improving production efficiency.

The design of the heat-conducting shell 211 and the inner shell 212 ensures that heat may be effectively transferred from the heating element 100 to the exterior of the hand warmer 200, thereby enhancing overall heating performance and helping to distribute heat uniformly across the device surface. The heating element 100 is bonded to the heat-conducting shell 211 via an adhesive layer, and the material of the heat-conducting shell 211 may be aluminum alloy, metal, or other heat-conductive materials. The design of the heating cavity 213 effectively isolates the heating element 100 from the internal structure of the housing 210, ensuring that heat is concentrated within a specific region, thereby improving heating efficiency and uniformity. The control board 220 is accommodated within the mounting cavity 214, keeping it separated from the heating element 100, thereby effectively preventing the high temperatures generated by the heating element 100 from directly affecting the control board 220. In this way, the stable operating temperature of the control board 220 is maintained, thereby preventing high temperatures from damaging electronic components and improving the reliability and service life of the control board 220.

In the embodiments of the present disclosure, the hand warmer 200 further includes a battery 240, which is placed inside the mounting cavity 214 and electrically connected to the control board 220 to supply power to the hand warmer 200.

Furthermore, the hand warmer 200 further includes a flexible member 230, which is sandwiched between the heating element 100 and the outer wall of the inner shell 212. The heating element 100 includes a temperature sensor 150, abutting against the flexible member 230.

Specifically, the flexible member 230 is disposed within the heating cavity 213, with one side connected to the outer wall of the inner shell 212 and the opposite side connected to the first coil of the heating element 100. This arrangement lifts the heating element 100, ensuring that it is tightly pressed against the heat-conducting shell 211, thereby enhancing the overall heat conduction efficiency. In addition, the flexible member 230 provides a soft contact surface, enabling the flexible member 230 to make soft contact with the temperature sensor 150, thereby serving as a buffer to prevent mechanical wear on the temperature sensor 150 caused by the temperature sensor 150 directly contacting the outer wall of the inner shell 212, so as to extend its service life. The flexible member 230 may be made of foam, silicone pads, or other flexible materials with cushioning properties. The way of the flexible member 230 being connected to the heating element 100 and the outer wall of the inner shell 212 may be implemented through adhesive bonding, friction fitting, or other methods.

Finally, it should be noted that the above embodiments are provided to illustrate the technical solutions of the present disclosure and are not intended to limit them. Although the present disclosure has been described in detail with reference to the aforementioned embodiments, those skilled in the art will understand that modifications may be made to the technical solutions described in the aforementioned embodiments, or equivalent replacements may be made to some of the technical features; such modifications or replacements do not cause the corresponding technical solutions to deviate from the spirit and scope of the technical solutions of the present disclosure.

What is claimed is:

1. A heating element for a hand warmer, comprising: a base body, at least three wiring terminals, a first coil, and a second coil;
wherein the first coil and the second coil are fixedly spaced apart on the base body, and the at least three wiring terminals are electrically connected to two ends of each of the first coil and the second coil.

2. The heating element according to claim 1, wherein the base body is configured in a plate-like shape, with the first coil and the second coil located on opposite surfaces of the base body.

3. The heating element according to claim 2, wherein the base body comprises a first surface and a second surface opposite the first surface, the first surface comprises a connection region and a heating region connected to the connection region, the first coil is distributed in the heating region, and the at least three wiring terminals are fixed in the connection region;
the at least three wiring terminals comprise a first terminal and a common terminal, wherein the first terminal and the common terminal pass through the first surface and the second surface to connect the two ends of the second coil, and the second coil is arranged in a corresponding heating region on the second surface facing the heating region and in a corresponding connection region on the second surface facing the connection region, while avoiding positions where the first terminal and the common terminal are located.

4. The heating element according to claim 3, wherein the second coil on the corresponding connection region extends serpentine along a first direction, and the second coil on the corresponding heating region extends serpentine along a second direction, with the first direction perpendicular to the second direction.

5. The heating element according to claim 4, wherein the second coil comprises a first segment and a second segment connected to the first segment, the first segment is connected to the first terminal, the second segment is connected to the common terminal, the first segment and the second segment are symmetrically arranged along the second direction.

6. The heating element according to claim 4, wherein the first coil comprises a third segment and a fourth segment extending serpentine along the second direction, with the third segment and the fourth segment distributed and connected along the first direction;
the heating element further comprises a temperature sensor, disposed on the first surface; wherein one of the two ends of each of the first coil and the second coil and one of two ends of the temperature sensor are connected to the common terminal.

7. The heating element according to claim 6, wherein the at least three wiring terminals further comprise a second terminal and a third terminal, the third segment is connected to the second terminal, and the fourth segment is connected to the common terminal, and the two ends of the temperature sensor are connected to the third terminal and the common terminal.

8. The heating element according to claim 1, wherein a surface of at least one of the first coil or the second coil is covered with an adhesive layer.

9. A hand warmer, comprising a housing, a control board, and a heating element;
wherein the heating element comprises: a base body, at least three wiring terminals, a first coil, and a second coil;
wherein the first coil and the second coil are fixedly spaced apart on the base body, and the at least three wiring terminals are electrically connected to two ends of each of the first coil and the second coil;
wherein the heating element and the control board are both arranged within the housing, and the heating element is electrically connected to the control board.

10. The hand warmer according to claim 9, wherein the base body is configured in a plate-like shape, with the first coil and the second coil located on opposite surfaces of the base body.

11. The hand warmer according to claim 10, wherein the base body comprises a first surface and a second surface opposite the first surface, the first surface comprises a connection region and a heating region connected to the connection region, the first coil is distributed in the heating region, and the at least three wiring terminals are fixed in the connection region;
the at least three wiring terminals comprise a first terminal and a common terminal, wherein the first terminal and the common terminal pass through the first surface and the second surface to connect the two ends of the second coil, and the second coil is arranged in a corresponding heating region on the second surface facing the heating region and in a corresponding connection region on the second surface facing the connection region, while avoiding positions where the first terminal and the common terminal are located.

12. The hand warmer according to claim 11, wherein the second coil on the corresponding connection region extends serpentine along a first direction, and the second coil on the corresponding heating region extends serpentine along a second direction, with the first direction perpendicular to the second direction.

13. The hand warmer according to claim 12, wherein the second coil comprises a first segment and a second segment connected to the first segment, the first segment is connected to the first terminal, the second segment is connected to the common terminal, and the first segment and the second segment are symmetrically arranged along the second direction.

14. The hand warmer according to claim 12, wherein the first coil comprises a third segment and a fourth segment extending serpentine along the second direction, with the third segment and the fourth segment distributed and connected along the first direction;
the heating element further comprises a temperature sensor, disposed on the first surface; wherein one of the two ends of each of the first coil and the second coil and one of two ends of the temperature sensor are connected to the common terminal.

15. The hand warmer according to claim 14, wherein the at least three wiring terminals further comprise a second terminal and a third terminal, the third segment is connected to the second terminal, the fourth segment is connected to the common terminal, and the two ends of the temperature sensor are connected to the third terminal and the common terminal.

16. The heating element according to claim 9, wherein a surface of at least one of the first coil or the second coil is covered with an adhesive layer.

17. The hand warmer according to claim 9, wherein the housing comprises a heat-conducting shell and an inner shell connected to the heat-conducting shell, the heat-conducting shell covers an outer wall of the inner shell, the heat-conducting shell and the inner shell enclose to define a heating cavity, the heating element is disposed within the heating cavity, the inner shell defines a mounting cavity, the heating cavity is in communication with the mounting cavity, and the control board is disposed within the mounting cavity, with the at least three wiring terminals facing the inner shell.

18. The hand warmer according to claim 17, wherein the hand warmer further comprises a flexible member, disposed within the heating cavity and sandwiched between the heating element and the outer wall of the inner shell, and the heating element comprises a temperature sensor, abutting against the flexible member.

19. The hand warmer according to claim 17, wherein the hand warmer further comprise a battery, placed inside the mounting cavity and electrically connected to the control board.

20. The heating element according to claim 6, wherein the temperature sensor is electrically connected to a control circuit, and the temperature sensor is configured to monitor a temperature of the heating element and adjust a power of the heating element through the control circuit;
in response to the temperature reaching a target temperature, low power is output;
in response to the temperature being below the target temperature, intermediate power is output; and
in response to the temperature still not reaching the target temperature after a certain period of intermediate power output, high power is output.
